(19) United States
(12) Patent Application Publication
(10) Pub. No.: US 2020/0406263 A1
(43) Pub. Date: Dec. 31, 2020

(54) MICROFLUIDIC CHIP, MICROFLUIDIC LAB-ON-CHIP, FABRICATION METHOD OF ONE SUCH CHIP AND ANALYSIS METHOD

(71) Applicant: NANOBIOSE, Sainte Hélène du Lac (FR)

(72) Inventors: Sarah MILGRAM, Crets En Belledonne (FR); Damien FLEURY, Barberaz (FR)

(73) Assignee: NANOBIOSE, Sainte Hélène du Lac (FR)

(21) Appl. No.: 16/956,632

(22) PCT Filed: Dec. 21, 2018
(86) PCT No.: PCT/FR2018/053524

§ 371 Date: Jun. 22, 2020

(30) Foreign Application Priority Data
Dec. 21, 2017 (FR) 1762895
Dec. 21, 2017 (FR) 1762896

(51) Int. Cl. B01L 3/00; G01N 33/68

(52) U.S. Cl. CPC B01L 3/502776; G01N 33/6863; B01L 2200/10; B01L 2300/0816; B01L 2300/0864; B01L 3/502707

(57) ABSTRACT

The microfluidic chip includes; at least one inlet channel connected to at least one well, each well being associated with an outlet channel, at least one analysis chamber connected to at least one well, an analysis surface including collection elements capturing biomarkers present in the liquid originating from the well and representative of the cellular response of a biological sample contained in the well. A liquid flow circulates from the well to the analysis surface including the collection elements. The liquid flowrate is between 0.1 μL/min and 2 mL/min and flows in laminar manner in the analysis chamber. The chip forms part of a microfluidic lab-on-chip provided with a flow generator applying several different liquids.

### BACKGROUND OF THE INVENTION

The invention relates to a microfluidic chip, a microfluidic lab-on-chip, a fabrication method of a microfluidic chip and an analysis method.

### STATE OF THE ART

To detect, identify and monitor the response of cells to stimuli of biological (viruses, bacteria, etc.), chemical (medication, etc.) or mechanical nature (application of a flow through the cells, of a pressure, a shear stress, etc.), a cytokine profile can be established. To do this, the nature of the cytokines secreted by the cells has to be known, as does their quantity and also the secretion kinetics of the cytokines.

At the present time, the techniques and equipment available for performing this type of study are complex and require numerous experimental steps resulting in these approaches being of little use from an industrial or quasi-industrial point of view. The cells first have to be cultured before the result of a reaction can be collected and analysed. Analysis will then involve several marking, washing, development and analysis steps. These steps are both long and costly. The result is only accessible several hours, or even several days, after the cell biology experiment was performed.

Therefore, to reduce the time required to run the analyses and the cost of the latter, the quantity of analysed samples and their exposure time are reduced to the strict minimum. However, when the quantity of samples and/or the analysis times are reduced too far, this can lead to results that give a poor reflection of the real response of the immune system as the equipment and/or protocols are not well suited.

To overcome this problem, different devices have been developed. According to the approach proposed in the document entitled “Reconfigurable microfluidic device with integrated antibody arrays for capture, multiplexed stimulation and cytokine profiling of human monocytes”, Vu et al., Biomicrofluidics 9, 04415 (2015), several cell populations are captured and organised in two dimensions on specific areas of an analysis surface. A perfusion system mounted on the analysis surface enables multiplexing, i.e. it is chosen to be able to simultaneously observe the cytokine response generated by several types of biochemical stimuli after the perfusion. The cytokines produced in response to the different stimuli are captured on other specific areas of the analysis surface and developed by means of specific reagents. Capture of the cells by their membrane receptors and 2D organisation of the latter creates a cell culture environment substantially removed from the actual physiological conditions.

In another direction of improvement, in the publication entitled “Integrated proteomic and transcriptomic investigation of the acetaminophen toxicity in liver microfluidic biochip” (PLoS One, 2011. 6(8): p. 212-68), Prot et al. propose to use microfluidics for culturing liver cells in a three-dimensional environment with a continuous perfusion of nutrients and toxic molecules. The analyses performed a posteriori enable the toxicity of the tested molecules to be quantified in more precise and dependable manner than under the 2D culture conditions without perfusion. The results obtained in 3D were in fact closer to those observed on laboratory animals.

Furthermore, in the publication entitled “High-Content Quantification of Single-Cell Immune Dynamics” (Cell Reports, Volume 15, 2016), Junkin et al. disclose a microfluidic device in which several biological tests can be run in parallel and in which the cells are placed under perfusion so as to perform dynamic analyses. The microfluidic system is coupled to a fluorescence microscope to observe the response of several individualised cells, i.e. cells isolated from one another, in real time.

These microfluidic devices endeavour to improve the conditions of analysis of the immune response of a cell culture. These improvements are however not sufficient to meet the expectations of the market which is keen to acquire an inexpensive device enabling complex analyses to be made and providing fast and dependable results.

### OBJECT OF THE INVENTION

One object of the invention consists in proposing a microfluidic chip enabling the response of a cell sample to be ascertained by performing complex analyses combining a more physiological cellular environment and continuous multi-parameter detection of biomarkers, and providing a result that is able to be used more quickly than with the solutions proposed by the prior art.

The chip is remarkable in that it comprises:
a first well designed to receive a biological sample, the substrate defining side walls and at least a bottom or top wall of the first well, the first well opening onto the surface on one side of the substrate,
a first inlet channel designed to supply the first well with a first liquid, the first inlet channel being terminated by a first inlet opening into the first well,
a first outlet channel having a first end formed by a first outlet of the first well to drain the first liquid,
a first analysis chamber having a first analysis inlet connected to the first well by means of the first outlet channel and a drain outlet opening into the first analysis chamber and distinct from the first analysis inlet, the first analysis chamber having a bottom wall formed by the substrate,
   wherein the first inlet and the first outlet of the first well define a first axis different from and not parallel to a second axis defined by the first analysis inlet and the drain outlet of the first analysis chamber,
   and wherein the first analysis chamber is closed off by an analysis wall comprising collection elements configured to capture at least one biomarker of the first liquid flowing in the first analysis chamber, the analysis wall being assembled removable with respect to the substrate.

In advantageous manner, the first well is closed by means of a covering layer distinct from the analysis wall so as to enable the analysis wall to be dismantled independently from the covering layer.

In one development, the covering layer is assembled in removable manner with respect to the surface of the substrate.

In a particular embodiment, the covering layer defines at least one through hole forming a ring surrounding the outlet of the first outlet channel, the ring defined in the covering layer completely or partially forming the side walls of the first analysis chamber, the surface of the substrate forming a bottom wall of the first analysis chamber.

In a preferential configuration, the side walls of the first analysis chamber are partially formed in the substrate, the first analysis chamber being formed by a first cavity opening onto the surface of the substrate, the covering layer defining a ring surrounding the first cavity and partially forming the side walls of the first analysis chamber.

In an alternative embodiment, the side walls of the first outlet channel are formed by a groove in the surface of the substrate, the covering layer closing off the first well and the first outlet channel up to the first analysis chamber.

In an advantageous configuration, the first outlet channel is defined by a groove formed in the covering layer closing off the first well.

In preferred manner, an additional channel is connected to the first outlet channel between the outlet of the first well and the first analysis chamber. The additional channel is advantageously formed by an additional groove in the surface of the substrate and/or in the covering layer.

It is advantageous to provide for the first analysis chamber to comprise first and second analysis inlets respectively defining first and second liquid inlet directions into the first analysis chamber. The first and second analysis inlets are arranged in such a way that the first and second liquid inlet directions are secant in the first analysis chamber.

Preferentially, the chip comprises a drain connected to the drain outlet of the first analysis chamber and first and second septums configured to tightly close the first inlet channel and the drain.

In another development, the distance separating the analysis wall and the bottom wall of the first analysis chamber is less than 2 mm.

It is a further object of the invention to provide a lab-on-chip that is compact while remaining particularly efficient.

The microfluidic lab-on-chip is remarkable in that it comprises:
a microfluidic chip according to one of the foregoing embodiments, the microfluidic chip comprising a covering layer closing off the first well and an analysis wall closing off the first analysis chamber in removable manner, the analysis wall being provided with collection elements configured to capture at least one biomarker of the liquid flowing in the first analysis chamber and originating from the first well,
a flow generator connected to the first inlet channel of the microfluidic chip, the flow generator being configured to emit at least a first liquid flow designed to pass through at least the first well filled by a cell sample and the analysis chamber, the first liquid flow having a flowrate comprised between 0.1 μL/min and 2 mL/min so that the liquid flow circulating in the first analysis chamber is laminar in at least one half of the first analysis chamber comprising the drain outlet.

In one development, the flow generator is connected to an additional channel of the microfluidic chip and the flow generator is configured to:
apply a first flow on the first inlet channel so as to feed the first well during a first period, the first flow comprising a first liquid containing at least nutrients,
apply an additional flow on the additional channel, the additional flow comprising reagents configured to react with biomarkers characteristic of a cellular response of the biological sample contained in the first well, the collection elements being configured to capture and/or react with the biomarkers.

It is a further object of the invention to provide a method for fabricating a chip that is easy to implement.

The method is remarkable in that it comprises the following successive steps:
providing a microfluidic chip according to one of the foregoing embodiments,
closing off the first well by means of a covering layer that at least partially forms the side walls of the first analysis chamber,
closing off the first analysis chamber by means of an analysis wall comprising collection elements configured to capture constituents of the liquid flowing in the first analysis chamber.

Another object of the invention consists in proposing a method for performing analysis of a cell culture by means of a microfluidic chip. The method enables complex analyses to be made, in fast and efficient manner, in order to ascertain the response of a cell culture with respect to several types of stimuli (chemical, biochemical, biological or pharmaceutical substance, mechanical stress, radiation, material, etc.).

For this purpose, the cell culture analysis method comprises:
providing a microfluidic chip comprising:
   a first well filled with a cell sample emitting a first cellular response containing at least one biomarker,
   a first analysis chamber having a first analysis inlet fluidly connected to the first well by means of a first outlet channel and a drain outlet opening into the first analysis chamber and distinct from the first analysis inlet,
   collection elements arranged on a wall of the first analysis chamber, the collection elements being configured to capture at least one biomarker representative of the first cellular response flowing from the first well to the first analysis chamber;
applying a first liquid flow feeding the first well so that the first cellular response is transported from the first well to the drain outlet through the first analysis chamber, said at least one biomarker flowing in the first analysis chamber to be captured by the collection elements,
analysing the collection elements.

According to a particular embodiment of the invention, the method comprises providing of at least one developing reagent configured to react with said at least one biomarker representative of the first cellular response captured by the collection elements, said at least one developing reagent passing through the first analysis chamber simultaneously or subsequent to the passage of said at least one biomarker in the first analysis chamber, analysis of the collection elements being performed by searching for said at least one developing reagent.

In preferred manner, the method comprises applying a second flow on an additional inlet of the microfluidic chip, the additional inlet opening into the first outlet channel via a first junction situated between the first well and the first analysis chamber so that the second flow enters the first analysis chamber, the second flow comprising said at least one developing reagent so that said at least one developing reagent does not have any contact with the cell sample.

According to a specific embodiment, the method comprises filling of the first outlet channel, after the first junction, alternately by the first flow comprising said at least one biomarker and by the second flow comprising said at least one developing reagent.

According to an alternative embodiment, the first junction is associated with a mixer configured to mix the first flow with the second flow in the first outlet channel after the first junction.

In another development, the microfluidic chip comprises a second well comprising an additional cell sample emitting a second cellular response with at least one additional biomarker, the second well being fluidly connected to the first analysis chamber by a second outlet channel joining the first outlet channel before the first analysis chamber, the flow generator supplying the first and second wells so as to convey the biomarker and the additional biomarker respectively representative of the first and second cellular responses to the first analysis chamber and to deliver first and second laminar flows inside the first analysis chamber, the biomarker and the additional biomarker being present respectively in the first and second laminar flows.

In a particular embodiment, the first well is filled with the cell sample before the first well is closed by a covering layer, the covering layer at least partially forming the side walls of the first analysis chamber.

In advantageous manner, the first analysis chamber is closed by means of an analysis wall comprising the collection elements.

Preferentially, the analysis method comprises dismantling of the analysis wall to perform analysis of the collection elements outside the first analysis chamber.

In a particular embodiment, the chip is defined in a substrate, the substrate defining a bottom wall and a side wall of the first well and a groove forming the first outlet channel, and the analysis method comprises closing of the first well and first outlet channel in tightly sealed manner by means of a removable covering layer.

According to an alternative embodiment, the covering layer defines an opening forming a closed ring around the first analysis chamber and, after the covering layer has been installed, the method comprises installation of an analysis surface closing the first analysis chamber in tightly sealed manner, the analysis surface comprising the collection elements.

According to a specific embodiment, the side walls of the first outlet channel are at least partially formed in the covering layer.

### BRIEF DESCRIPTION OF THE DRAWINGS

Other advantages and features will become more clearly apparent from the following description of particular embodiments of the invention given for non-restrictive example purposes only and represented in the appended drawings, in which:

FIG. 1 represents a microfluidic chip according to a first embodiment,

FIG. 2 represents a microfluidic chip according to a second embodiment,

FIG. 3 represents an exploded view of a microfluidic chip according to an embodiment of the invention,

FIG. 4 represents a microfluidic lab-on-chip using the chip of FIG. 3 after the different parts have been assembled, and a flow generator, the flow being represented schematically by arrows at the level of the inlets,

FIG. 5 represents another embodiment of a microfluidic chip,

FIGS. 6 and 7 represent other embodiments of a microfluidic chip.

### DETAILED DESCRIPTION

A microfluidic chip can advantageously be used in the field of biomedical and/or biological analyses, for example to understand the impact of chemical, biological and/or mechanical stresses on the immune system. It is also possible to monitor the impact of a material or of a radiation.

As illustrated in the different figures, microfluidic chip 1 comprises at least a first well 2a designed to receive a biological sample partly composed of living cells, for example—tissues, a biopsy, organoids and/or a eukaryote or prokaryote cell culture. For example purposes, chip 1 is considered to be associated with a cell culture in the rest of the description. First well 2a comprises a bottom wall, a side wall and a top wall. First well 2a is a cell culture well, i.e. it is designed to contain cells to be analysed or it is designed to contain cells of a cell culture.

First well 2a has a first inlet opening into first well 2a and a first outlet opening out from first well 2a and distinct from the first inlet. In this way, it is possible to have a liquid flow passing through first well 2a so as to be close to in vivo conditions. First well 2a is made from a substrate A that defines the side walls of the well and advantageously the bottom and/or top wall of well 2a. Depending on the embodiments, substrate A can be formed by a single material or by several materials, for example by a stack of several different layers. Substrate A is preferentially monobloc.

Microfluidic chip 1 further comprises a first analysis chamber 3a having a first analysis inlet. First analysis chamber 3a is designed for performing analysis of molecules present in the liquid coming from well 2a which are representative of the cellular response.

Microfluidic chip 1 comprises a first inlet channel 4a designed to feed first well 2a. First inlet channel 4a opens into first well 2a via the first inlet. First inlet channel 4a is designed to be connected to a flow generator supplying microfluidic chip 1 with a liquid or several different liquids. First inlet channel 4a is terminated by the first inlet.

The chip further comprises a first outlet channel 5a designed to drain the first liquid from well 2a. First outlet channel 5a has a first end formed by the first outlet of well 2a. Inlet channel 4a, first well 2a and first outlet channel 5a are arranged in series in the direction of flow of the fluid.

First analysis chamber 3a is connected to first well 2a by means of first outlet channel 5a. First analysis chamber 3a has a first analysis inlet arranged at a first end of chamber 3a and a first drain outlet located at a distance from the first analysis inlet. The first analysis inlet and first drain outlet define the through flow of the liquid inside first analysis chamber 3a. In advantageous manner, the first drain outlet is located at the opposite end from the first analysis inlet.

First analysis chamber 3a is dissociated from first well 2a to facilitate analysis and possibly so as not to disturb the cells present in first well 2a with reagent products used to observe and/or analyse the cellular response. Analysis chamber 3a receives the liquid coming from well 2a comprising elements representative of the cellular response or biomarkers via first outlet channel 5a so that analysis chamber 3a continuously receives information representative of the cellular response. Analysis chamber 3a is preferentially separated from first well 2a by an outlet channel that represents a volume of liquid of less than 1004 so that the information provided by first well 2a reaches analysis chamber 3a quickly. This configuration makes it possible to monitor the reaction of the cells in well 2a in time by limiting the disturbance introduced by analysis of the cellular response.

Analysis chamber 3a is closed off by an analysis wall 6. Analysis wall 6 is in contact with the liquid coming from well 2a flowing through the analysis chamber. Analysis wall 6 presents an analysis surface on which collection devices or probes 6a are installed to capture certain elements of the liquid flowing in analysis chamber 3a to enable them to be detected.

Well 2a has small dimensions so as to reduce the quantity of cells used and the quantity of reagents to be used. However, to obtain or measure a sufficient signal to be able to perform the analyses, a configuration of well 2a has to be provided facilitating stimulation of the cells and/or recovery of the products resulting from the reaction of the cells without having to apply a high pressure in the chip and in particular in well 2a. In advantageous manner, the volume of first well 2a is less than 1 mL which enables the size of the well to be limited without penalising measurement. Also in advantageous manner, the volume of first well 2a is greater than 5 μl.

It is particularly advantageous to use a well enabling a three-dimensional cell culture scaffold to be installed so as to be close to in vivo conditions. In addition to the three-dimensional organisation of the cells which is more physiological, the advantage of culture scaffolds or three-dimensional matrices is to increase the culture surface per volume unit of the well. This makes it possible to have more cells secreting biomarkers and therefore more biomarkers per volume unit thereby reducing the cellular response detection limit. It is particularly advantageous to use a textured or porous scaffold to increase the culture surface. Each of the three dimensions of well 2a is more than 100 μm and preferably less than 10 mm. The height of the well is advantageously greater than or equal to the hydraulic radius of the well.

The inventors propose judiciously placing the liquid inlet and the liquid outlet so as to ensure a homogeneous flow in most of the well in order to have a cellular response close to in vivo conditions.

The first inlet channel defines a first flow direction of the liquid. The first outlet channel defines a second flow direction. The inventors observed that to achieve an enhanced efficiency in stimulation of the cell culture, it is advantageous to provide for the first and second flow directions not to be aligned. In this way the nutrients and molecules to be tested are better distributed inside first well 2a.

When the inlet and outlet channels have an axis of revolution, the flow directions are defined by the axes of revolution.

The offset of the flow directions limits the stagnation areas of liquid in well 2, i.e. the dead volumes. The proportion of the biological sample functioning in static state is reduced thereby improving the quality of the cellular signal obtained. This configuration ensures that the products coming from the inlet are placed in the presence of most of the cells.

Advantageously, the inlet and outlet of first well 2a define a first axis different from and not parallel to a second axis defined by the inlet and outlet of analysis chamber 3a. The inlet and outlet of well 2a are advantageously arranged so that the flow inside well 2a runs mainly along a longitudinal axis of the well, for example its axis of revolution. In this configuration, the cells arranged in well 2a are in contact with the liquid flow thereby achieving in particular an enhanced cellular response and therefore a more dependable analysis with a small quantity of cells used.

This configuration enables a three-dimensional cell culture well 2 to be coupled with a two-dimensional analysis chamber 3a. Analysis chamber 3a presents a ratio between the chamber volume and the surface of the side walls of chamber 3a that is substantially higher than the ratio between the volume of the well and the surface of the side walls of well 2a. This configuration maximizes the surface of the top and bottom walls of the analysis chamber per volume unit. This enables the use of an analysis surface to be optimized by promoting contact of the molecules to be detected with collection devices 6a present on the analysis surface. The main advantage of a flat or substantially flat analysis surface is its compatibility with conventional optic detection instruments. Furthermore, such an analysis surface enhances the possibility of multiplexing detection, i.e. the possibility of multiplying the number of different molecules detected by multiplying the number of different collection devices 6a present on the analysis surface.

The first inlet and first outlet can open into a bottom or top wall, or onto a side wall of first well 2a near the bottom or the top.

The bottom wall of well 2a is separated from the top wall of well 2a by a first distance along the longitudinal axis. It is advantageous to provide for the first inlet to be separated from the first outlet by a distance at least equal to half the first distance along the longitudinal axis. To facilitate stimulation of the cells and retrieval of the cellular response, in even more advantageous manner, the inlet is separated from the outlet by a distance at least equal to 75% of the first distance along the longitudinal axis.

In preferential manner, the first outlet comes into contact with a bottom wall or top wall of well 2a when the latter is closed. As an alternative, the first outlet is formed in a top wall or bottom wall and preferentially along an axis that is not perpendicular to the longitudinal axis.

In a particular embodiment, the first outlet and first inlet are located on side walls of well 2a. This embodiment is particularly advantageous for observing the cell culture by microscopy.

In an advantageous embodiment, the liquid inlet takes place at the bottom of well 2a. It is advantageous to have an inlet channel 4a having a longitudinal axis which is not parallel, for example perpendicular to the longitudinal axis of outlet channel 5a. This embodiment does however result in difficulty of observation of the cell culture by optic means.

In an advantageous embodiment, in a section plane perpendicular to the height of well 2a, the well presents transverse dimensions comprised between 100 μm and 10 mm to limit the volume of well 2a and also the size of well 2a. As the volume of well 2a is small, the same is true of the quantity of cells.

In advantageous manner, well 2a is a cavity having a volume of revolution. The cross-section of the cavity can be square, rectangular, circular, ovoid or of any shape. The cross-section is observed in a first plane that is advantageously parallel to the top and/or bottom surface of the substrate defining the well. Advantageously, well 2 is a straight circular cylinder which provides a configuration closer to in vivo conditions.

In a particular embodiment, the walls of well 2a or at least a part of the walls of well 2a are covered by a coating configured to enable the cells to adhere and to be kept inside well 2a. Depending on the configurations, only the side walls are covered with a coating configured to enable the cells to adhere or as an alternative the side walls and top wall are covered by the coating. It is further possible to provide for the bottom wall to also be covered by a coating enabling the cells to adhere.

As an alternative or as a complement, the cells are cultured in one or more three-dimensional scaffolds placed inside well 2a so as to create a three-dimensional cell culture structure. The three-dimensional scaffolds can be matrices or gels depending on the type of cells studied. Performing three-dimensional cell cultures makes it possible to come close to in vivo conditions and therefore to obtain a response that is close to the actual response of a living organism.

First analysis chamber 3a comprises a bottom wall, side walls and a top wall. First analysis chamber 3a collects the liquid coming from well 2a and the constituents of this liquid are captured on analysis surface 6a. The constituent or constituents are representative of the cellular response, i.e. of the response of the cell culture.

Preferentially, analysis chamber 3a is configured to present a larger width than that of outlet channel 5a coming from well 2a.

The increased width is measured in a direction perpendicular to the direction of transit of the liquid flow in analysis chamber 3a and preferably in a horizontal direction in operation.

In a particular embodiment, the depth of analysis chamber 3a is identical to the depth of outlet channel 5a. The depth is measured perpendicular to the bottom wall of the analysis chamber and/or to the analysis surface. In an alternative embodiment, the depth of analysis chamber 3a is larger than the depth of outlet channel 5a. In another embodiment, the depth of analysis chamber 3a is smaller than the depth of outlet channel 5a.

In a particular embodiment, the bottom wall of first analysis chamber 3a is coplanar with the bottom of first outlet channel 5a thereby making it easier to obtain a flow in the analysis chamber. As an alternative, the bottom wall of chamber 3a presents an obstacle, for example a step or ramp facilitating contact of the liquid with the analysis surface. The step or ramp is defined so that the liquid flow encounters an obstacle which forces the liquid flow to move towards the opposite wall of analysis chamber 3a, i.e. the analysis surface containing collection devices 6a.

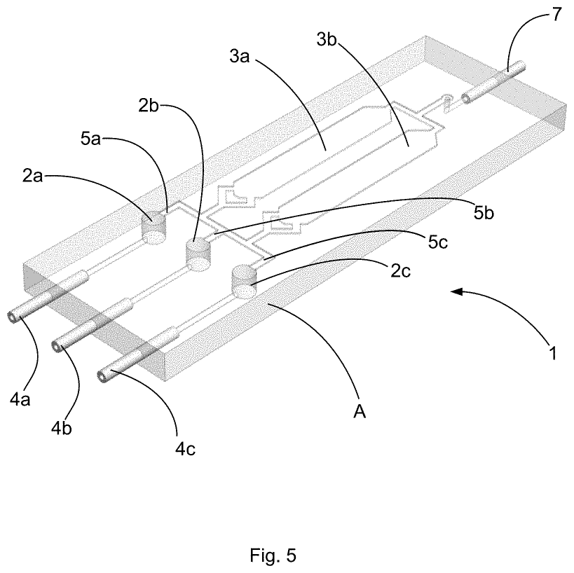

When analysis chamber 3a comprises first and second analysis inlets or at least two analysis inlets as illustrated in FIG. 5, it is particularly advantageous to provide for two inlets to be configured to inlet two liquid flows to analysis chamber 3a in two secant inlet directions. In this way, the two liquid flows cross one another inside chamber 3a. In advantageous manner, the two inlets are fed via one and the same channel and provide the same liquid preferentially comprising the biomarkers representative of the cellular response and/or a developing reagent.

As an alternative, the two liquids are different. It is for example possible to provide a liquid flow comprising biomarkers and a liquid flow comprising a developing reagent.

This crossing of the flows has the effect of increasing the thickness and width of the liquid flow in analysis chamber 3a thereby facilitating a contact between the liquid containing the cellular response and the analysis surface configured to capture elements representative of the cellular response. It is particularly advantageous to use this configuration when the thickness of analysis chamber 3a is greater than the thickness of outlet channel 5a and/or when the width of analysis chamber 3a is greater than the width of outlet channel 5a.

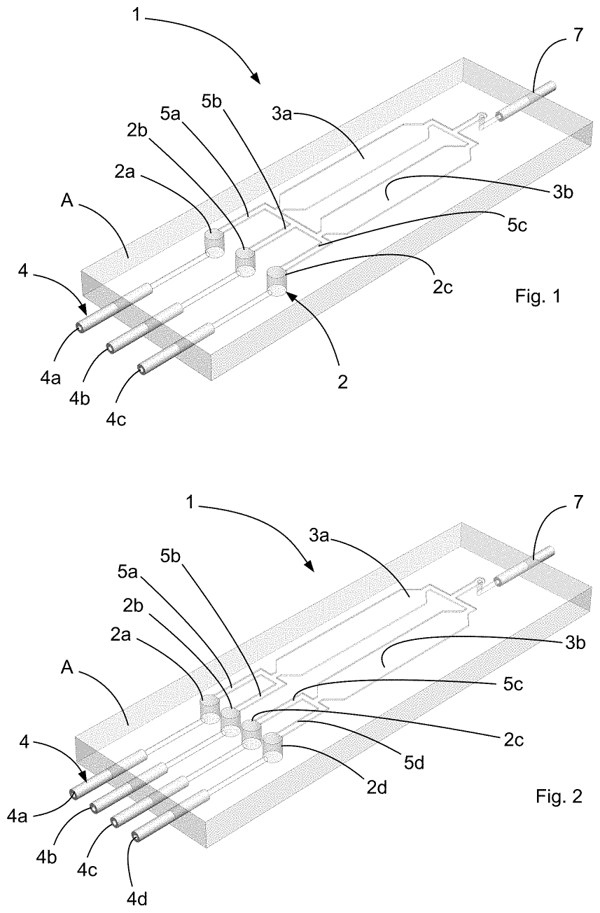

In a particular embodiment illustrated in FIGS. 1, 2 and 5, first well 2a and first analysis chamber 3a are formed in a substrate A. First analysis chamber 3a is formed by a cavity opening onto a surface of substrate A.

In a particular embodiment, well 2a does not have a bottom wall or top wall formed by substrate A so as to form a blind cavity in the substrate. In this configuration, it is easier to insert a three-dimensional scaffold receiving the cell culture. Well 2a is closed off by means of covering wall 8, advantageously in tightly sealed manner. In other words, first well 2a is formed in a substrate A and comprises a removable top wall formed by a covering layer 8 covering one surface of substrate A. Well 2a can be easily filled and then closed by covering layer 8.

In the exemplary embodiment illustrated in the different figures, if substrate A comprises several wells, covering layer 8 is common to all the wells 2 as this makes chip 1 easier to fabricate. However it is also possible to provide for each well 2 to be closed off by a specific covering layer 8 to adapt to the cell culture or for a single covering layer 8 to be able to be used for several wells 2. Depending on the embodiments, a covering layer 8 closes off one or more wells 2.

As an alternative, covering layer 8 is used to close the well or wells but it is not removed to fill the wells. It is then possible to inject the cell culture through covering layer 8, for example by means of a syringe. Such a configuration makes fabrication and especially use of the chip easier.

In yet another embodiment, well 2 has a bottom wall and a side wall formed in a substrate A. The well is closed by covering layer 8 that is distinct from substrate A and is not removable. The cells and their culture scaffold are then placed in well 2 by means of inlet channel 4 and/or outlet channel 5 or by injection through covering layer 8 of well 2, for example by means of a syringe.

In advantageous manner, microfluidic chip 1 is formed in monolithic manner in a substrate that defines first well 2a, inlet channel 4a, outlet channel 5a and possibly first analysis chamber 3a. It is also advantageous to form the channel connecting the outlet of analysis chamber 3a with a drain 7 inside substrate A.

In order to guarantee the dependability of the measurements, microfluidic chip 1 is made from a biocompatible material providing an optimal viability and a cellular functionality close to in vivo conditions. The material of microfluidic chip 1 can be chosen from polymethyl methacrylate, polydimethylsiloxane, polyether ether ketone, cyclic olefin copolymer, polycarbonate, polystyrene, a mixture containing Exo-1,7,7-trimethylbicyclo[2.2.1]hept-2-yl acrylate and tricyclodecane dimethanol diacrylate for example MED610™, stainless steel, glass, etc. It can also be made from a 3D printing material having the property of being biocompatible, such as MED610™.

In a configuration that is not illustrated, outlet channel 5a is completely formed in the substrate. As an alternative, in the illustrated configuration, outlet channel 5a is not closed in its top part and substrate A advantageously defines a groove that connects the top part of well 2 with analysis chamber 3a. Covering layer 8 closes off first outlet channel 5a.

In advantageous manner, covering layer 8 is covered by an adhesive material so as to be able to be easily fixed against the surface of substrate A. Depending on the embodiments, the adhesive material can cover well 2a or not. If the adhesive material covers well 2a, the adhesive material is biocompatible as it comes into contact with the cell culture.

In an advantageous embodiment, covering layer 8 closes off well 2a and outlet channel 5a. Covering layer 8 defines an opening for access to analysis chamber 3. Advantageously, covering layer 8 forms a closed ring around the side wall of first analysis chamber 3a. Covering layer 8 continuously covers well 2a, outlet channel 5a and the circumference of analysis chamber 3 thereby facilitating installation of analysis wall 6 which closes off analysis chamber 3a in tight manner. Analysis wall 6 forms the top of analysis chamber 3a presenting the analysis surface with collection devices 6a. In a particular configuration that is not illustrated, covering layer 8 defines a non pass-through groove forming the bottom wall of first analysis chamber 3a.

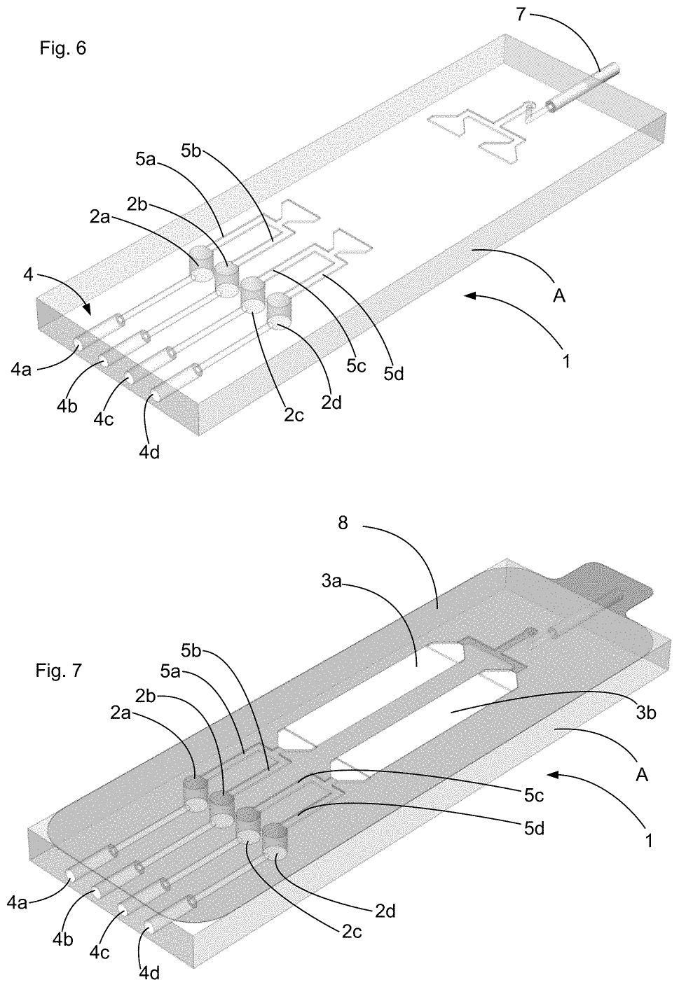

In a preferential embodiment illustrated in FIGS. 6 and 7, substrate A comprises a hole preferably blind hole to define well 2a. Substrate A on the other hand does not define or completely define the side walls of first analysis chamber 3a. The latter are formed by means of covering layer 8. In the illustrated embodiment, the bottom wall of well 2a, the side walls of well 2a, the bottom and side walls of channel 5a and the bottom wall of analysis chamber 3a are formed in substrate A.

In the embodiment of FIG. 7, the thickness of analysis chamber 3a is defined by the thickness of covering layer 8.

In advantageous manner and as illustrated in FIGS. 6 and 7, outlet channel 5a is flared on entry to analysis chamber 3a. Outlet channel 5a has side walls that allow the flow to broaden before entering analysis chamber 3a. Preferentially, broadening of outlet channel 5a is performed in such a way as to achieve the same width as that of analysis chamber 3a defined by the side walls of covering layer 8.

The end of outlet channel 5a is formed by the end of the groove in substrate A so that the liquid reaches the surface of substrate A. The step or ramp formed by the end of the groove forces the liquid to wet the analysis surface thereby making measurement easier to perform. It is particularly advantageous to provide for analysis surface 6 to be facing outlet channel 5a to enhance the establishment of a laminar liquid flow in analysis chamber 3a.

The bottom of analysis chamber 3a is arranged on at least two different planes one of which corresponds to the top surface of substrate A.

As illustrated in FIGS. 6 and 7, analysis chamber 3a is formed by depositing covering layer 8 on substrate A. It is therefore possible to modulate the characteristics of analysis chamber 3a in simple manner by adjusting the dimensions of the opening formed in covering layer 8 and/or by adjusting the thickness of covering layer 8. This configuration is particularly advantageous as it enables the dead volume in analysis chamber 3a to be limited.

In a particular embodiment that is not illustrated, covering layer 8 is arranged on the surface of substrate A in order to close well 2a and outlet channel 5a. Covering layer 8 is monobloc and has a blind cavity defining analysis chamber 3a.

Analysis wall 6 defines the analysis surface with its collection elements or probes 6a configured to capture constituents of the liquid flowing in first analysis chamber 3a. Analysis wall 6 is installed in removable manner. The analysis surface containing probes 6a is separated from substrate A by covering layer 8 to perform analysis of the cellular response. Installation and removal of analysis wall 6 can then be performed without disturbing well 2a, i.e. avoiding removing covering layer 8 closing well 2a.

The analysis surface is covered by the collection elements. Collection elements 6a are configured to fix one or more biomarkers, i.e. the components of the fluid originating from well 2a representative of the cellular response that is to be analysed. Collection elements 6a can also be configured to fix the developing reagent thereby achieving positive development monitoring. The analysis surface can comprise areas with positive development monitoring and/or areas with specific biomarker captors, as well as visible positioning markers when acquisition of the signal or of the measurement takes place.

The biomarker is a measurable biological characteristic linked to a process. The biomarker can be a molecule produced, secreted or modified by the cells of the cell sample. The biomarker can be a chemical molecule, a fragment of DNA or RNA, a metabolite, a sugar, a lipid, a protein or a fragment of protein specific to the cellular response. Probes 6a can comprise for example antibodies, proteins, lipids, aptamers, sugars, peptides, fragments of immunoglobulin or oligonucleotides. In analysis chamber 3, the constituents of the cellular response detected or biomarkers are preferentially cytokines. Continuous detection of the cytokines enables a kinetic profile to be established. It is advantageous to install several probes 6a reacting to the same biomarker or biomarkers to improve the quality of the analysis.

By using an analysis wall 6 that is removable with respect to the rest of analysis chamber 3, it is possible to adapt detection to the cellular reaction in the course of the experiment. The use of a removable wall 6 is particularly advantageous when the analysis surface comprises several different detection probes configured to distinguish different elements of the cellular response, i.e. to react to different biomarkers or a cellular secretion. Removable analysis wall 6 enables the chamber to be opened to replace analysis surface 6 and, if required, probes 6a.

For example, if the cellular response changes with time, it is possible to change the probes of the analysis surface to match the probes to the response, for example according to the nature of the cytokines or their concentration, so that the measurement can follow the dynamics of the cellular response. In this way, saturation of the signal is avoided.

Probes 6a may also become impaired with time. In order to monitor the cellular response over a relatively long period, it is advantageous to replace analysis wall 6 comprising worn probes by a new analysis wall 6 comprising new probes.

By using a removable analysis wall, it is possible to install and dismantle the analysis surface without disturbing the cells present in well 2a.

In an alternative embodiment, analysis wall 6a is fixed to covering layer 8. Analysis wall 6a and covering layer 8 can be formed from identical or different materials.

If the chip comprises several analysis chamber, it is advantageous for covering layer 8 to form a continuous ring around each analysis chamber 3. Preferentially, covering layer 8 comes into direct contact with substrate A. Covering layer 8 forms a sealing part ensuring continuity of covering between the well, the outlet channel and the circumference of the analysis chamber. Covering layer 8 also forms a seal by making the surface of the substrate match the surface of analysis wall 6.

In one embodiment, covering layer 8 defines an opening that corresponds exactly to the shape of each opening existing in the substrate and that defines an analysis chamber 3a so as not to disturb the liquid flow running from the inlet of analysis chamber 3a to the outlet of analysis chamber 3a.

The height of analysis chamber 3a is at least partially defined by the thickness of covering layer 8. To ensure a homogeneous diffusion of the flow in analysis chamber 3a, the height of the side wall of analysis chamber 3a can be less than half the difference between the width of analysis chamber 3a and the width of outlet channel 5a. In even more advantageous manner, the thickness of covering layer 8 is chosen such that the distance separating analysis wall 6 and the bottom of first analysis chamber 3a is less than 2 mm.

In an advantageous embodiment, the groove formed by channel 5a and analysis chamber 3a belong to one and the same plane from the outlet of well 2 to the outlet of analysis chamber 3a.

Covering layer 8 can be transparent at optic wavelengths to be able to observe the cell cultures present in well 2 by optic microscopy, such as fluorescence microscopy when covering layer 8 covers well 2.

Depending on the embodiments, analysis wall 6 can be deposited directly on covering layer 8 or these two layers are separated by at least one bonding layer 9. Bonding layer 9 enables a better mechanical connection to be obtained between analysis wall 6 and covering layer 8.

To form an efficient seal, the material forming covering layer 8 or bonding layer 9 can be chosen from polyethylene, polytetrafluoroethylene, polyurethane, a glass fabric impregnated with polytetrafluoroethylene, silicone, or polydimethylsiloxane. It is also possible to use an adhesive of acrylic type. Bonding layer 9 can be formed by a gel, a paste, or a foam.

To enhance the contact between the liquid coming from well 2 and the analysis surface, the thickness of the assembly formed by covering layer 8 and bonding layer 9 must be smaller than or equal to the difference between the radius or half-width of analysis chamber 3a and the radius or half-width of channel 5a.

It is advantageous to provide for the wall comprising the reagent coating or the wall opposite the wall comprising the reagent coating to be transparent to the analysis signal so as to facilitate measurement in the course of reaction. This makes it possible to make a measurement in the course of reaction.

It is advantageous to apply a pressure on the removable analysis wall to improve the sealing of chip 1. The pressure can be applied by a mechanical compression system such as clamps, magnets or electromagnets, a suction, or a pneumatic system.

To facilitate analysis of the constituents of the cellular response in first analysis chamber 3a, the inventors observed that it is particularly advantageous to use a developing reagent. The developing reagent will react with certain constituents emitted by the cell culture so as to fix markers on the constituents which will be fixed on probes 6a of first analysis chamber 3a. The developing reagent can also react with the probes themselves to perform for example positive monitoring or internal development calibration.

Different embodiments can be envisaged to make the cellular response constituents coming from the well react with a developing reagent.

The cellular response constituents or biomarkers present in the liquid and outlet from well 2a are captured by probes 6a of the analysis surface.

In one configuration, capture of the biomarkers gives rise to an analysis signal enabling the cellular response to be monitored. However, in a very large number of cases, capture of the biomarkers does not give rise to an analysis signal and a specific developing reagent is added and used as monitoring element of the biomarker characteristic of the cellular response to be monitored.

In order to improve monitoring of the cellular response, it is particularly advantageous to insert the developing reagent before the liquid is inlet to analysis chamber 3a, i.e. before the inlet of the analysis chamber, either by mixing the developing reagent with the liquid leaving the well or by alternating the flow of reagents and liquid leaving the well. As a variant, the developing reagent is added on the analysis surface after the analysis wall has been separated from the rest of analysis chamber 3a.

It is possible to perform analysis of the cellular response directly in analysis chamber 3a if the developing reagent fixes itself on the biomarker itself fixed on probes 6a and induces an analysable signal, and provided that at least a part of the chamber is transparent to the induced signal. Otherwise, it is advantageous to provide for the analysis wall to be able to be extracted from analysis chamber 3a to perform analysis of collection elements 6a.

If the developing reagent is inlet to the chip, it is advantageous to insert the developing reagent after the outlet of well 2a so as not to disturb the cell culture. The chip advantageously comprises an additional channel connected to first outlet channel 5a between the outlet of first well 2a and first analysis chamber 3a at the level of a first junction. In this way, the developing reagent can be mixed with the liquid outlet from first well 2a without disturbing the cell culture.

The developing reagent can be placed in contact with biomarkers by mixing or by alternative supply of the biomarkers and then of the developing reagent in the analysis chamber.

To enable analyses to be formed in real time by microscopy, the wall presenting the analysis surface can be transparent at optic wavelengths. It can also be made from a non auto-fluorescent material to enable fluorescence microscopy to be used as analysis technique. The analysis wall is preferably made from glass, plastic or composite material and covered or not by a thin metal, polymer or chemical functionalisation layer.

It is further possible to provide for measurement of the cellular response not to be able to be performed directly in analysis chamber 3a. This particular case can occur when wall 6 of the chamber is not transparent to the radiation used to perform measurement or when the radiation used to perform measurement may impair the biomarkers situated in chamber 3a so that the first measurement distorts the subsequent measurements.

In all these specific cases, it is advantageous to remove analysis wall 6 comprising the reagent coating from chamber 3a, to fit a new analysis wall 6 with a reagent coating and to place the reagent coating brought into contact with the liquid containing the biomarkers in a measuring device, for example by optic means, by luminescence, absorbance or fluorescence.

In advantageous manner, the chip comprises an additional channel 4b/5b connected to first outlet channel 5a between the outlet of first well 2a and first analysis chamber 3a. Outlet channel 5a is designed to be fed by an additional flow which is advantageously a liquid containing a developing reagent.

As indicated in the foregoing, outlet channel 5a can be defined in substrate A. It is also possible to provide for the walls of outlet channel 5a to be partially or totally formed in covering layer 8. The same can be the case for additional channel 5b.

Advantageously, additional channel 4b/5b comprises a second inlet channel 4b connected to first outlet channel 5a by means of a mechanical energy dissipater and a second outlet channel 5b. First outlet channel 5a and second outlet channel 5b join one another before reaching analysis chamber 3a.

In preferential manner, the mechanical energy dissipater is configured to define a pressure loss equal to more or less 20% of the pressure loss defined by first well 2a. Depending on the embodiments, the liquids originating from first outlet channel 5a and second outlet channel 5b are mixed with one another or not.

It is particularly advantageous to provide for the mechanical energy dissipater to be formed by a second well 2b identical to first well 2a. When chip 1 comprises first and second inlet channels 4a and 4b, first and second wells 2a, 2b and first and second outlet channels 5a and 5b, it is advantageous to provide for first and second inlet channels 4a and 4b to define a first plane parallel to and offset from a second plane defined by first and second outlet channels 5a, 5b.

According to a particular embodiment, the microfluidic chip comprises a mixer (not shown) configured to mix at least two laminar liquid flows upstream from the mixer and to deliver a laminar flow on outlet from the mixer. The mixer is advantageously placed between the outlet of first well 2a and the inlet of analysis chamber 3a. The mixer comprises a first inlet fed by first well 2a and a second inlet fed by second well 2b or by an inlet distinct from the inlet feeding first well 2a. The mixer is configured to mix two flows present on inlet and to provide at least one flow on outlet. The outlet flow corresponds to the mixture of the inlet flows.

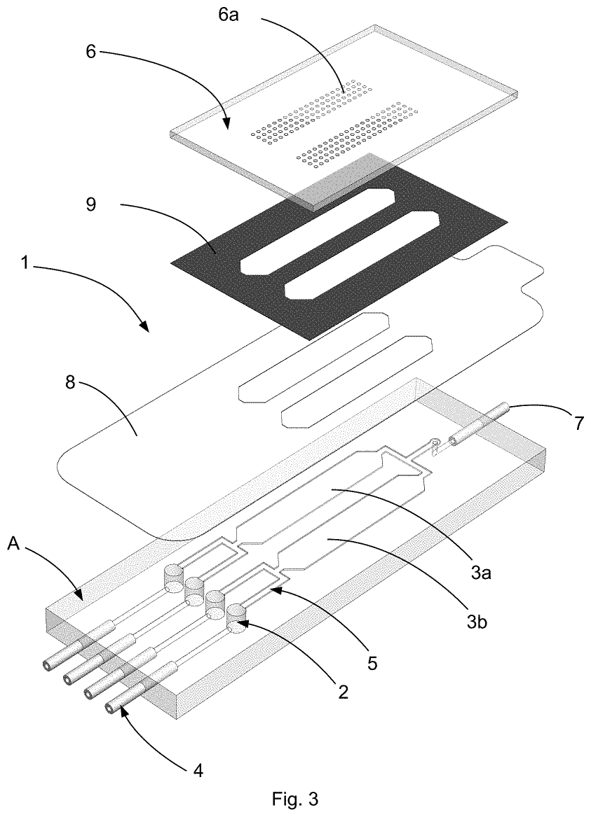

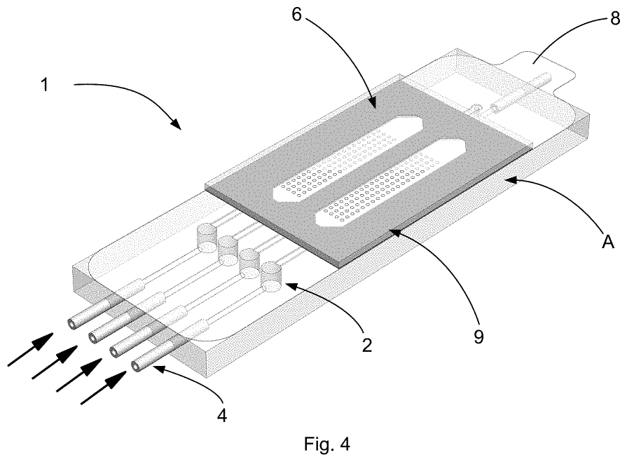

As illustrated in FIGS. 1, 2, 3, 4, 5, 6 and 7, microfluidic chip 1 can comprise a plurality of cell culture wells denoted 2a, 2b, 2c and 2d connected to one or more analysis chambers, here two chambers 3a and 3b. The inlet of each well 2 is connected to an inlet channel 4 and the outlet of each well 2 is connected to an outlet channel 5. An analysis chamber 3 can be connected to one or more different wells 2 by means of one or more outlet channels 5.

As well 2 occupies a small surface, it is possible to provide a large quantity of wells 2 in a small surface thereby making it easier to fabricate a compact analysis device. Miniaturisation of the wells also enables the number of wells per device to be multiplied thereby multiplying the number of biological samples analysed per analysis.

According to a first particular embodiment illustrated in FIGS. 1 and 5, a microfluidic chip 1 comprises first, second and third inlet channels 4a, 4b and 4c respectively connected to first, second and third wells 2a, 2b and 2c arranged fluidly in parallel. First, second and third wells 2a, 2b and 2c are respectively connected to first, second and third outlet channels 5a, 5b and 5c. In one embodiment, one of the wells can be replaced by an additional channel. In the illustrated embodiment, it is advantageous to provide for well 2b to be replaced by the additional channel.

First and second outlet channels 5a and 5b join one another before reaching first analysis chamber 3a. Second and third outlet channels 5b and 5c join one another before reaching second analysis chamber 3b. The two analysis chambers are arranged fluidly in parallel between wells 2a, 2b and 2c and a drain 7.

In the embodiment illustrated in FIGS. 1 and 5, the three outlet channels are connected to a single transverse channel from which the inlets of the two analysis chambers 3a and 3b branch off. First analysis chamber 3a is connected to receive the liquid from first outlet channel 5a and the liquid from second outlet channel 5b. Second analysis chamber 3b is connected to receive the liquid from third outlet channel 5c and the liquid from second outlet channel 5b. In this configuration, when the pressure applied in the three wells 2a, 2b and 2c is identical, first chamber 3a receives a flow coming from first well 2a and a flow coming from second well 2b, and second chamber 3b receives a flow coming from third well 2c and a flow coming from second well 2b. The information contained in second outlet channel 5b is present in the two chambers arranged in parallel.

In the illustrated example, the two analysis chambers 3a and 3b have an outlet which is finally connected to drain 7.

Analysis chambers 3 and drain 7 off from the substrate are advantageously situated in two different parallel planes.

According to an alternative embodiment of the microfluidic chip represented in FIGS. 2, 3, 4, 6 and 7, a chip 1 can comprise four inlets 4a, 4b, 4c and 4d respectively connected to four cell culture wells 2a, 2b, 2c and 2d. Chip 1 also comprises two analysis chambers 3a and 3b each connected to two cell culture wells 2. First and second wells 2a and 2b are connected to first analysis chamber 3a whereas third and fourth wells 2c and 2d are connected to second analysis chamber 3b. There again, one or two wells can be replaced by additional channels to provide reagents.

The two analysis chambers 3a/3b are finally connected to a drain 7 to enable the analysed products to be drained off. In the illustrated embodiment, drain 7 is common.

In the embodiments illustrated in the different figures, microfluidic chip 1 comprises a plurality of wells 2 each connected to an inlet channel 4. Each well 2 has its own outlet channel 5. Outlet channels 5 of several wells 2 join one another before entering an analysis chamber 3. At least one analysis chamber 3 is connected to at least two wells 2 of microfluidic chip 1.

In the advantageous embodiments illustrated, the outlets of wells 2 are connected to one another before reaching analysis chamber 3 so that the different flows coming from the plurality of wells 2 enter analysis chamber 3 via the same inlet. By using a laminar flow in analysis chamber 3, it is possible to have several liquid flows coming from several wells 2 in the same analysis chamber 3 without the flows being mixed with one another. It is particularly advantageous to have a Reynolds number less than 100 to further reduce the probability of mixing of the liquids in the analysis chamber. In advantageous manner, at least two liquids from at least two different wells are present in the analysis chamber.

By judiciously placing probes 6a at several locations of analysis chamber 3 on the analysis surface in a direction perpendicular to the liquid flow inside chamber 3, it is possible to analyse the different biomarkers originating from well 2. The different biomarkers are present at the same time in analysis chamber 3 and are separated from one another in a direction perpendicular to the direction connecting the inlet and outlet of chamber 3, i.e. in a direction perpendicular to the flow direction of the liquid in analysis chamber 3. It is particularly advantageous to place several probes 6a on the analysis surface in a direction perpendicular to the direction of flow in analysis chamber 3 to analyse the different cellular responses separately.

In this configuration, it is possible to perform analysis of several cell samples placed in different wells 2 with a single analysis chamber 3 without the cellular responses being mixed up. Each liquid flow runs from the inlet to the outlet and conveys biomarkers characteristic of the response of the cell sample of a well 2.

The analysis surface is analysed by a measurement device that can then measure the different cellular responses on the same analysis surface.

As an alternative, it is also possible to have an analysis chamber connected to a single well itself fed by a single inlet channel.

In particularly advantageous manner, inlet channels 4 are closed by a septum. In this embodiment that is not illustrated, first inlet channel 4a has a first end opening into first well 2a and a second end that is closed by a tight membrane. The tight membrane is configured to form a septum that can be pierced for example by a needle. When the needle is removed, the membrane becomes tight again. This configuration enables the tightness of first inlet 4a to be preserved thereby preventing first well 2a from being contaminated. The sterility of the cell culture can be preserved in spite of a large number of connection/disconnection steps of chip 1 with a flow generator. The tightness in the rest of the chip can be obtained with other means.

A tight membrane can also be used as septum at the level of outlet or outlets 7. In this case, one or more needles can be used to pass through the septum and achieve the fluidic communication with a tank that recovers the liquids.

When the inlets and drains are closed by a septum, the microfluidic chip is tight. This configuration enables the culture area and the analysis area, which form a monobloc element, to be moved in complete safety.

The flow generator enables one or more liquids to be injected into the different inlets of chip 1 by means of needles passing through each septum.

Microfluidic chip 1 is advantageously associated with a flow generator, for example a pump, to form a lab-on-chip. The pump is preferentially configured so that the liquid flows in analysis chambers 3 are laminar. Advantageously, the liquid flows between the outlet of well 2 and the inlet of analysis chamber 3 are also laminar. Preferably, the flow generator is configured so that the liquid flows between the outlet of well 2 and the outlet of analysis chamber 3 present a Reynolds number less than 100. It is even more advantageous to provide for the flow in well 2 to also be laminar.

To ensure efficient transport of the elements present in the liquid from well 2 to the analysis surface, the flow conditions of the fluid are configured to promote transport of the liquids by convection rather than by diffusion. It is advantageous to choose flow conditions such that the Péclet number in the pipe connecting well 2 to analysis chamber 3 and advantageously in analysis chamber 3 is less than 1. The Péclet number is a dimensionless number representing the ratio of the advective transport rate over the diffusive transport rate.

The lab-on-chip is advantageously configured so that the liquid flowrate is comprised between 0.1 μL/min and 2 mL/min which enables a laminar flow to take place in analysis chamber or chambers 3. By using such a flow, fixing of the biomarkers on the probes is enhanced. Preferably, the liquid flowrate is comprised between 0.1 μL/min and 1 mL/min. The value of the liquid flowrate advantageously corresponds to a mean value, for example a mean value over one minute. It is also possible to use a liquid flow in the form of pulses with a flowrate value comprised between 1 mL/min and 5 mL/min, the flowrate value preferably being greater than 4 mL/min. The pulses have durations of less than 10 seconds.

The markers characteristic of the modification of the response of the cells in time are to be found in the liquid flow outlet from well 2 that is inlet to analysis chamber 3. With such a flow, the modification of the response of the cells can be monitored in real time in analysis chamber 3 and in particular between the well outlet and the inlet of analysis chamber 3.

The flow generator is connected to chip 1 to make a fluid transit from well 2 to analysis chamber 3. The flow generator can be a pressure controller, a syringe pump or other pump (not shown). The flow generator can be configured to supply the chip with fluid either continuously or in periodic or aperiodic manner.

When a continuous feed supply is involved, a liquid flow is present continuously through well 2a and analysis chamber 3a. The flowrate is not zero and can be variable in time. The flowrate can change in periodic or aperiodic manner. In the other embodiments, the flowrate varies with a period at zero flowrate and a period at non-zero flowrate. During the period at non-zero flowrate, the flowrate can be constant or variable.

The flow generator is configured to deliver at least one liquid chosen from a feed liquid comprising nutrients, a growth liquid, or a molecule to be tested, for example a chemical or biological stimulus which can be a medicinal product. It is further possible to deliver a supply of developing reagents. The molecules to be tested can for example be chemical or biochemical molecules so as to observe the impact of these molecules on the cellular response. The developing reagents can for example be fluorescent markers so as to be able to use fluorescence microscopy when performing the analyses.

In a particular embodiment, well 2a is fed continuously by a first liquid flow. The first liquid flow can contain nutrients. The first liquid flow can be completed by a second liquid flow having a different composition from that of the first liquid flow.

When the composition of the liquid changes in time at the inlet of well 2a, it is possible to provide for the first flow to mainly comprise nutrients during a first period. During a second period, the flow comprises molecules to be analysed. Preferably, the molecules to be tested are conveyed by means of a liquid containing a culture medium. The culture medium contains nutrients but it can also contain salts and buffers. It is also possible to provide for the culture medium to contain predefined reagents if the cells are to be analysed directly. The liquid flowrate can be constant regardless of the composition of the liquid.

Supplying the cell cultures by means of a continuous flow enables the nutrition of the cell cultures to be improved compared with a static nutritional input. The experimental conditions are therefore close to in vivo conditions.

It is also possible to vary the concentration of biological or chemical stimuli so as to simulate the cell exposure conditions following administration of a medicinal or other product. The concentration of stimuli can increase during a first phase and then decrease during a second phase.

The cell cultures are subjected to chemical, biochemical, physical or mechanical stimuli in order to evaluate their response, i.e. their viability or mortality and the nature of the molecules or biomarkers they secrete. In the study of a mechanical stimulus, the pressure and/or flowrate may change in time and the chemical composition is constant or variable.

The lab-on-chip is therefore extremely advantageous as it enables the cellular secretions to be analysed in real time and genuine kinetic studies to be performed close to in vivo conditions. Furthermore, the extensive surface of analysis chamber 3a enables a large number of biomarkers to be detected in cost-effective manner.

This type of lab-on-chip is particularly advantageous as it enables a very large number of different analysis methods to be used. In the case where the chip comprises several wells arranged in parallel, it is possible to choose the feed supply of the different wells differently both as far as the composition and flowrate and the modification of these two parameters in time are concerned. The analyses can be performed in parallel resulting in an appreciable time saving.

It is possible to feed all the wells with the same solution.

As an alternative, it is possible to dissociate the fluid feeding the wells between at least two wells so as to compare the cellular reaction between the wells. For example, it is possible to compare the cellular response between a flow comprising chemical and/or biological stimuli and a flow devoid of chemical and/or biological stimuli. It is further possible to change the concentration of nutrients or the concentration of chemical and/or biological stimuli between the wells to determine the influence of these different parameters on the cellular response.

In general manner, the analysis method of the cellular response of a biological sample can present itself in the following manner.

The method comprises a first step of providing a microfluidic chip 1. It is possible to use the chip described above, but it is also possible to use a different chip.

Microfluidic chip 1 comprises a first well 2a filled by a cell sample emitting a first cellular response. The cell sample can be deposited manually in first well 2a using a pipette or micropipette.

The chip further comprises a first inlet channel 4a designed to feed first well 2a. The first inlet channel opens into an inlet of first well 2a.

The chip also comprises a first analysis chamber 3a having a first analysis inlet fluidly connected to first well 2a by means of a first outlet channel 5a and a drain outlet opening into first analysis chamber 3a and distinct from the first analysis inlet.

The chip further comprises collection elements 6a arranged on a wall of first analysis chamber 3, collection elements 6a being configured to capture at least one biomarker representative of the first cellular response flowing from first well 2a to the drain outlet of first analysis chamber 3a.

The method further comprises provision of a flow generator configured to apply a first liquid flow feeding first well 2a so that the first cellular response is conveyed from first well 2a to the drain outlet through first analysis chamber 3a. The biomarker is then transported from first well 2a to the drain outlet of the first analysis chamber so that said at least one biomarker is captured by collection elements 6a. Collection elements 6a are analysed to monitor the cellular response of the cell sample.

The flow generator is configured to provide liquid flows at least to first inlet channel 4a.

In advantageous manner, the well is filled by a liquid containing nutrients before the cell culture is inlet to the well. In a particular embodiment, inlet channels 4 of the chip are connected to the flow generator and the flow generator is configured so that a liquid supplies well 2 with a first liquid advantageously comprising nutrients. In a particular organisation mode, the flow generator is configured to fill the well completely without filling the analysis chamber. In advantageous manner, the generator is configured to fill a plurality of wells with the same flowrate for each well.

Once the well has been filled by a liquid, a cell culture is inlet to the well. For example a cell culture matrix is inlet to the well. It is particularly advantageous to place the cell culture in the well after the well has been filled by a fluid thereby avoiding leaving the cell culture in the open air resulting in drying of the cell culture. In advantageous manner, at least channels 4 are filled before the cell culture is inlet to the well.

In a specific case, closing of well 2 is performed at the same time as fitting of a first seal around analysis chamber 3. The seal leaves analysis chamber 3 accessible.

In a particular method of use, closing of well 2 is performed before the cells are placed in well 2. The cells will then be placed in well 2 by injection via inlet channel 4 or through covering layer 8, for example by means of a syringe.

Analysis chamber 3 can then be closed by means of analysis wall 6. Once analysis chamber 3 has been tightly closed, the flow generator can fill analysis chamber 3 with the liquid. Preferentially, the flow generator fills analysis chamber 3 from a flow coming from well 2. In advantageous manner, the flow generator is configured to fill well 2 and then analysis chamber 3 up to drain 7.

The analysis method advantageously comprises a feed step of first well 2a comprising the cell culture with a first liquid having a first composition and comprising nutrients. First well 2a is fed with a first flowrate and a first pressure.

In a second step, first well 2a can be fed with a second liquid comprising a second composition. First well 2a is fed with a second flowrate and a second pressure, at least one parameter chosen from the second composition, the second flowrate and the second pressure being respectively different from the first composition, the first flowrate and the first pressure. The cell culture emits biomarkers in response to the second liquid. The feed with first and second liquids is configured so that the biomarkers reach analysis chamber 3. The biomarkers are captured by probes 6a and the difference of response between the first liquid and the second liquid for the same cell culture in the same chip can be compared.

By modifying the pressure and/or the flowrate, it is possible to simulate a mechanical stress on the cell culture. By modifying the composition of the nutrient medium it is possible to simulate chemical stress. However, it is particularly advantageous to add at least one molecule to the second liquid, i.e. the molecule to be studied. As an alternative, the molecule to be studied is present continuously. The constituents of the first liquid and of the second liquid can be identical and with different concentrations.

The biomarkers representative of the cellular response contained in the liquid originating from the cell culture wells are then captured on probes 6a present in analysis chamber 3. If necessary, a developing reagent is placed in contact with the biomarkers captured on probes 6a to highlight an analysable signal.

Advantageously, the method comprises provision of at least one developing reagent configured to react with the biomarker or biomarkers representative of the first cellular response captured by collection elements 6a. The developing reagent passes through first analysis chamber 3a simultaneously or subsequently to the passage of the first cellular response in first analysis chamber 3a. Analysis of collection elements 6a is performed by searching for the developing reagent.

When the microfluidic chip comprises a plurality of wells for example at least two, three or four wells, it is possible to use the inlet channels of the chip differently.

In one configuration, a first well 2a is filled by cells and a second well 2b is not filled by cells. First well 2a is subjected to a first liquid flow comprising nutrients and possibly to chemical or biological stimuli. Second well 2b then becomes an additional inlet opening into first outlet channel 5a via a first junction located between first well 2a and first analysis chamber 3a. The latter is subjected to a second flow of developing reagents. The second flow enters analysis chamber 3 without being in contact with the cell sample.

In order for analysis chamber 3a to be fed both by reagents coming from the additional inlet or from well 2b and by the liquid coming from cell culture well 2a, different embodiments are possible. The outlets of first well 2a and second well 2b join one another at the level of the first junction in a mixer so that the second flow of reagents mixes with the first flow of biomarkers in outlet channel 5a after the first junction. On outlet, the mixer delivers a liquid corresponding to the mixture of the two flows. There is then no risk of the reagents or the reagent inlet flowrate disturbing the cells of first well 2a.

As an alternative, the analysis chamber is fed alternately by well 2a and then by well 2b so that all the probes see the liquid containing the biomarkers of the cellular response and the liquid containing the developing reagents alternately.

For example, if the chip comprises three wells as illustrated in FIGS. 1 and 5, it is possible to associate a well and its inlet channel to a reagent flow. The other two wells are filled by cell cultures. The outlets of the wells containing cell cultures each join up with the outlet of the well associated with the developing reagent. There again, it is possible to provide for several wells to contain cells and for these wells to be fed with different compositions. The reagent flow is inlet to the central well and the end wells contain the cell cultures.

The well containing the cell culture is fed by a nutrient liquid. After a first waiting time, the cell culture is also fed by the molecule to be studied. Simultaneously to inlet of the molecule to be studied or subsequent to this inlet, the developing reagent is applied on the second inlet channel so that the developing reagent is inlet to the analysis chamber by mixing or in succession with the liquid coming from the cell culture wells. In advantageous manner, this chaining of steps is performed by the flow generator which comprises a control circuit. This configuration is particularly advantageous when the developing reagent is detrimental to the cell culture.

In another embodiment, the two wells 2a and 2b are filled by cell cultures. As previously, a liquid flow comprising nutrients is applied in each of wells 2. The liquid flow of at least one of wells 2 is then modified so as to input a molecule to be studied in each of wells 2. It is conceivable to input the molecule to be studied in one of the wells only.

The developing reagent can be applied on the analysis surface outside the chip after the analysis wall has been removed. This embodiment is particularly advantageous in combination with the chip illustrated in FIG. 2 where two wells are used to supply a single analysis chamber. The embodiment illustrated in FIGS. 1 and 5 enables different conditions to be compared using the condition applied in well 2b as reference.

Well 2b can also advantageously be associated with the reagent input, wells 2a and 2c then being used to study cell culture stimulation operating conditions. Each analysis chamber receives the same reagent and a specific cellular response coming from well 2a or well 2c. As an alternative, well 2b receives the cell culture and wells 2a and 2c are associated with different reagents and/or the two analysis chambers have different probes 6a.

The chip also enables two different cell cultures to be provided in two different wells the outlets of which join one another or not in a single analysis chamber.

The two wells are subjected to the same liquid flows, preferentially in simultaneous manner. It is then possible to observe the response of the two different cultures.

It is further possible to provide for two wells to be filled by two different cultures and for each culture to be subjected to different liquid flows. Integration on one and the same chip ensures that the other conditions studied are identical, for example as far as the temperature, humidity and/or atmosphere are concerned.

In an advantageous embodiment, the well is kept at a setpoint temperature, for example by means of an incubator. The setpoint temperature is advantageously comprised between 20 and 45° C., preferentially equal to 37° C. In a particular embodiment, the incubator is configured to apply the setpoint temperature when filling of the well takes place and when filling of the analysis chamber takes place. As an alternative, the incubator is configured to apply the setpoint temperature once the cell culture is inlet to the well. The incubator can also be configured to provide a controlled atmosphere with 5% per volume of CO₂ and a humidity of at least 90% per volume.

In order to obtain study conditions that are closest to in vivo conditions, it is particularly advantageous to provide for the flow generator to supply the well and the analysis chamber when the chip is at the setpoint temperature.

1-25. (canceled)

26. Microfluidic chip comprising
a substrate defining:
a first well designed to receive a biological sample providing at least one biomarker, the substrate defining side walls and at least a bottom wall or top wall of the first well, the first well opening onto a surface on one side of the substrate,
a first inlet channel designed to feed the first well with a first liquid, the first inlet channel being terminated by a first inlet opening into the first well,
a first outlet channel having a first end formed by a first outlet of the first well to drain the first liquid,
a first analysis chamber having a first analysis inlet connected to the first well by means of the first outlet channel and a drain outlet opening out from the first analysis chamber and distinct from the first analysis inlet,
wherein the first inlet and the first outlet of the first well define a first axis different from and not parallel to a second axis defined by the first analysis inlet and the drain outlet of the first analysis chamber,
a covering layer closing the first well,
an analysis wall closing the first analysis chamber, the analysis wall comprising collection elements configured to capture the at least one biomarker of the first liquid flowing in the first analysis chamber, the analysis wall being assembled in removable manner with respect to the substrate and with respect to the covering layer so that the analysis wall can be dismantled independently from the covering layer.

27. Microfluidic chip according to claim 26, wherein the covering layer is assembled in removable manner with respect to the surface of the substrate.

28. Microfluidic chip according to claim 26, wherein the covering layer defines at least one through hole forming a ring surrounding an outlet of the first outlet channel, the ring defined in the covering layer completely or partially forming side walls of the first analysis chamber, the surface of the substrate forming a bottom wall of the first analysis chamber.

29. Microfluidic chip according to claim 26, wherein side walls of the first analysis chamber are partially formed in the substrate, the first analysis chamber being formed by a first cavity opening onto the surface of the substrate, the covering layer defining a ring surrounding the first cavity and partially forming the side walls of the first analysis chamber.

30. Microfluidic chip according to claim 26, wherein side walls of the first outlet channel are formed by a groove in the surface of the substrate, the covering layer closing off the first well and the first outlet channel up to the first analysis chamber.

31. Microfluidic chip according to claim 26, wherein the first outlet channel is defined by a groove formed in the covering layer closing off the first well.

32. Microfluidic chip according to claim 26, comprising an additional channel connected to the first outlet channel between the outlet of the first well and the first analysis chamber, the additional channel advantageously being formed by an additional groove in the surface of the substrate and in the covering layer.

33. Microfluidic chip according to claim 26, comprising an additional channel connected to the first outlet channel between the outlet of the first well and the first analysis chamber, the additional channel advantageously being formed by an additional groove in the surface of the substrate or in the covering layer.

34. Microfluidic chip according to claim 26, wherein the bottom wall of the first analysis chamber forms an obstacle fostering the first liquid to flow toward the analysis wall.

35. Microfluidic chip according to claim 26, comprising a drain connected to the drain outlet of the first analysis chamber and first and second septums configured to tightly close the first inlet channel and the drain.

36. Microfluidic lab-on-chip comprising:
a microfluidic chip according to claim 26, the microfluidic chip comprising an analysis wall closing off the first analysis chamber in removable manner, the analysis wall being provided with collection elements configured to capture at least one biomarker of the liquid flowing in the first analysis chamber and originating from the first well,
a flow generator connected to the first inlet channel of the microfluidic chip, the flow generator being configured to emit at least a first liquid flow designed to pass through at least the first well filled by a cell sample and the analysis chamber, the first liquid flow having a flowrate comprised between 0.1 μL/min and 2 mL/min so that the liquid flow circulating in the first analysis chamber is laminar in at least one half of the first analysis chamber comprising the drain outlet.

37. Microfluidic lab-on-chip according to claim 36, wherein the flow generator is connected to an additional channel of the microfluidic chip and wherein the flow generator is configured to:
apply a first flow on the first inlet channel so as to feed the first well during a first period, the first flow comprising a first liquid containing at least nutrients,
apply an additional flow on the additional channel comprising reagents configured to react with biomarkers characteristic of a cellular response of the biological sample contained in the first well, the collection elements being configured to capture and/or react with the biomarkers.

38. Method for fabricating a microfluidic chip comprising the following successive steps:
providing a microfluidic chip according to claim 26,
closing off the first well by means of a covering layer that at least partially forms the side walls of the first analysis chamber,
closing off the first analysis chamber by means of an analysis wall comprising collection elements configured to capture biomarkers of the liquid flowing in the first analysis chamber.

39. Analysis method of a cell sample comprising:
providing a microfluidic chip according to claim 26 wherein
the first well is filled by a cell sample emitting a first cellular response containing at least one biomarker;
the first analysis chamber does not contain a cell sample and has a first analysis inlet fluidly connected to the first well by means of the first outlet channel;
the collection elements are configured to capture at least one biomarker flowing from the first well to the first analysis chamber;
applying a first liquid flow feeding the first well so that the first cellular response is transported from the first well to the drain outlet through the first analysis chamber, said at least one biomarker flowing in the first analysis chamber to be captured by the collection elements;
analysing the collection elements;
wherein a monobloc substrate at least partially defines the first well and wherein the collection elements are assembled removable with respect to the first analysis chamber.
